(19) United States
(12) Patent Application Publication
(10) Pub. No.: US 2025/0282653 A1
(43) Pub. Date: Sep. 11, 2025

(54) METHOD FOR OPERATING A REVERSE OSMOSIS SYSTEM

(71) Applicant: B. Braun Avitum AG, Melsungen (DE)

(72) Inventors: Nino Lieb, Melsungen (DE); Philipp Odernheimer, Melsungen (DE)

(21) Appl. No.: 19/069,483

(22) Filed: Mar. 4, 2025

(30) Foreign Application Priority Data
Mar. 6, 2024 (DE) 10 2024 106 402.1

(51) Int. Cl. C02F 1/44; C02F 1/00; C02F 103/02

(52) U.S. Cl. CPC C02F 1/441; C02F 1/008; C02F 2103/026; C02F 2201/007; C02F 2209/005

(57) ABSTRACT

A reverse osmosis system, which includes a booster pump, a membrane module, and a ring line with a tapping point, is operated by: a) selecting a system-specific parameter and defining an optimized system reaction of the reverse osmosis system as a function of the system-specific parameter; b) iteratively modifying the system-specific parameter; c) measuring the system response of the reverse osmosis system to the modified system-specific parameter of the reverse osmosis system; optionally adjusting the changing of the system-specific parameter of the reverse osmosis system to achieve a system response closer to the optimized system response; e) when the optimized system reaction is reached within a predetermined threshold range, defining an optimized operating parameter and/or threshold value; and f) operating the reverse osmosis system with the optimized operating parameter and/or threshold value.

### CROSS-REFERENCE TO RELATED APPLICATIONS

This application claims priority under 35 U.S.C. § 119 to German Application No. 10 2024 106 402.1, filed on Mar. 6, 2024, the content of which is incorporated by reference herein in its entirety.

### FIELD

The present disclosure is directed to a method for operating a reverse osmosis system.

### BACKGROUND

Reverse osmosis systems are used in the medical field to produce water, e.g. for dialysis machines, which then perform dialysis treatment. During dialysis treatment, patients come into contact with a large amount of dialysis fluid. During the therapy, the dialysis fluid and the patient's blood flow through a dialyzer and are separated from each other within the dialyzer only by a semipermeable membrane. There, a transfer of the substances to be removed from the blood during dialysis therapy, such as urea, takes place from the blood across the semipermeable membrane into the dialysis fluid. Clinical studies show that contamination in dialysis water can contribute to acute and chronic problems and lead to severe complications in hemodialysis patients if, for example, they enter the patient's bloodstream. Therefore, water quality is a key factor in modern dialysis.

A device and a method suitable for a reverse osmosis system are known from U.S. Pat. No. 6,797,173 B1. A process chamber with an inlet, a low-pressure outlet and a high-pressure outlet is provided. A feed pump is used to increase the feed pressure for the process chamber.

A disadvantage of known reverse osmosis systems is that they are operated without taking into account the current ambient conditions, the material condition and the condition of the connected devices.

### SUMMARY

The present disclosure is therefore based on the objective of providing a method for operating a reverse osmosis system that enables optimized operation of a specifically installed reverse osmosis system and eliminates the disadvantages of the prior art.

This objective is solved by a method according to the present disclosure. The method for operating a reverse osmosis system, which comprises at least one booster pump, at least one membrane module and a ring line with at least one tapping point, has the following steps:
a) selecting at least one system-specific parameter of the reverse osmosis system and defining an optimized system reaction of the reverse osmosis system in dependence on the at least one system-specific parameter of the reverse osmosis system;
b) iteratively modifying the at least one system-specific parameter of the reverse osmosis system;
c) measuring the system reaction of the reverse osmosis system to the at least one modified system-specific parameter of the reverse osmosis system;
d) optionally adjusting the modification of the at least one system-specific parameter of the reverse osmosis system in order to achieve a system reaction that is closer to the optimized system reaction;
e) when the optimized system reaction is reached within a predetermined threshold range, defining at least one optimized operating parameter;
f) operating the reverse osmosis system with this at least one optimized operating parameter.

The present disclosure is based on the consideration that a reverse osmosis system should be operated as effectively and efficiently as possible. At the same time, it should be operated in a resource-saving manner. Although such a reverse osmosis system can be optimized by the manufacturer to a certain extent before delivery, the specific installations of the reverse osmosis systems can differ, for example due to replaced components, the age of components or the specific environmental conditions, so that the same initial configuration is not optimized for all reverse osmosis systems.

As has now been recognized, a reverse osmosis system can be operated in an optimized manner by optimizing system-specific operating parameters for the specifically installed reverse osmosis system, so that the reverse osmosis system can then be operated with these optimized parameters. This optimization of the parameters can be carried out directly after the installation of the system. However, it can also be carried out as needed or at regular intervals so that technical or material changes or the type of connected devices can be taken into account.

Advantageously the variation of the parameter(s) is carried out at a speed and/or step size such that the system reaction to the modified parameters has fully adjusted before the next variation of the parameters, so that the reverse osmosis is in a (dynamic) equilibrium before the next variation of the parameter(s) occurs.

In a first preferred embodiment of the method, the optimized system reaction corresponds to a threshold value of at least one measurable variable in the reverse osmosis system. A measurable variable here preferably denotes a system reaction measurable by means of at least one sensor, for example a pressure in a line.

Advantageously, a first pressure in the reverse osmosis system or downstream thereof at the start of the ring line is measurable and a second pressure is measurable at at least one tapping point.

In step a), the speed of the booster pump is selected as the system-specific parameter, whereby the optimized system reaction is present when the first pressure reaches a first pressure threshold value first or when the second pressure reaches a second pressure threshold value first, and whereby

in step b) the speed of the pressure-boosting pump is increased iteratively, and in step c) the first pressure and the second pressure are measured, and in step e) when the optimized system reaction is reached within the predetermined threshold range, i.e. when one of the two pressure threshold values is reached, the current pressure is set as the maximum pressure threshold value of the reverse osmosis system at the beginning of the ring line. As soon as one of the two threshold values is reached, the pressure at the beginning of the ring line is thus set as the new threshold value.

The beginning of the ring line refers to the area of the ring line where the permeate enters before flowing through the ring line to the tapping points. At each of the two locations, the pressures are measured while the booster pump increases its speed. As soon as one of the two pressure limits is reached, the process is terminated. The optimized value resulting from this procedure is applied to the pressure sensor and measured there.

If the pressure threshold is first reached at a pressure sensor at the beginning of the ring line, this measured pressure is used as the maximum pressure threshold. If the pressure threshold at the tapping point reaches the pressure threshold first, the pressure present at that time at the beginning of the ring line is used as the maximum pressure threshold.

This ensures that the reverse osmosis system can be operated at a maximum pressure, which is determined by the maximum permissible pressure for components of the reverse osmosis system or connected devices.

In a second preferred embodiment of the method, the optimized system response corresponds to an extremum, in particular a minimum, of an error measure dependent on the system-specific parameters. In particular, a minimum is defined if the error measure is defined as the sum of positive-definite summands.

Advantageously the reverse osmosis system has at least one control circuit with at least one controller with at least one controller parameter, where in step a) characteristic variables of the controller are selected as system-specific parameters, and where the optimized system reaction corresponds to a minimum of an error measure which depends on these characteristic variables, and where in step b) at least one of the characteristic variables is changed in an iteration, and in step c) the error measure is determined on the basis of the system response, and in step e) when the optimized system response is reached within the predetermined threshold range, the at least one current controller parameter is set as the optimized control parameter.

Advantageously, step d) is carried out by carrying out a gradient step method for changing at least one characteristic variable in steps b) and d). The gradient in a parameter space corresponds to the direction in the parameter with the greatest change. As a result, the method quickly moves to the extremum by following the gradient. It is thus considerably more effective than other optimization methods in which the parameters are essentially varied independently of one another.

In the gradient step method, a variable step size that depends on the rate of change of the error measure is advantageously used for the iteratively modified parameters.

In a preferred embodiment, the error measure is a weighted sum of the characteristic variables. The weighting is preferably realized by multiplication of the respective characteristic variable with a scale factor, whereby an adaptation to a sum of variables with different units can also be carried out.

The preferred characteristic variables are rise time, settling time and overshoot/undershoot. For rise time and settling time, the actual times from the start of the measurement to reaching the end point are measured. For the rise time, the time taken to rise from 10% to 90% of the final value is preferred. For the settling time, the time required for the process value to reach the final value within specified limits (e.g. +/−2%) is preferably measured. This value “penalizes” controllers that have low damping, since these controllers tend to oscillate more.

The at least one controller is preferably a PI controller, where the controller parameters are Kp and Ti.

In a preferred embodiment of the method, the method step d of adjusting the changing of the at least one system-specific parameter of the reverse osmosis system in order to achieve a system response that is closer to the optimized system response is a mandatory method step that is carried out in particular iteratively with the method steps b and c. This means in particular that each iteration of the process comprises the sequence of steps b, c and d, where in step d, for example, the step size of the parameter change is changed, this sequence being terminated at process step e of reaching the optimized system response within a predetermined threshold range and defining at least one optimized operating parameter and/or threshold value. This enables the optimized operating parameter and/or threshold value associated with the optimized system response to be found in a particularly targeted and effective manner.

In a preferred embodiment of the method, the method steps a to e are carried out in a pre-operation and/or intermediate operation, in which no consumer removes liquid at any of the tapping points. This pre-operation/intermediate operation is therefore relevant because in this way the time-invariant system, in particular the reverse osmosis system and ring line, can be evaluated and measured without disturbance variables (dialysis machines or other consumers). This is advantageous because otherwise the disturbance variables would be evaluated as part of the system characteristic, which would falsify the at least one iteration of the method.

In particular, the process steps a to e are preferably carried out in a preliminary operation, for example before the very first start-up, before the reverse osmosis is operated regularly, i.e. with consumers connected to the tapping points. This ensures that the reverse osmosis system operates optimally from the outset.

The process steps a to e are further preferably carried out in an intermediate operation at regular intervals and/or as required. This means that the regular operation of the reverse osmosis system is interrupted and steps a to e are carried out. In this way, changes in the reverse osmosis that occur over time and that can lead to modified optimized operating parameters can be taken into account. Changes that occur over time in the reverse osmosis system include, for example, a change in the system characteristics of the membranes. These can cause a higher pressure loss due to blocking and/or microbial phenomena, which in turn affects the system dynamics. Likewise, signs of wear and tear on components and/or altered fluid dynamics due to deposits in the device are noteworthy examples of a transformation of what is actually a static system.

It is advantageous to apply a test impulse to the reverse osmosis system in step c to measure the system reaction. The test impulse serves to bring the reverse osmosis into a system state that allows the determination of the corresponding (steady-state) system reaction.

For the test impulse, the reverse osmosis system can be started from the off state or an idle state and set to the value of the system-specific parameter, whereby the test impulse is terminated when the system reaction has reached a stationary end value or after a predefined time has elapsed.

During the test impulse, the reverse osmosis can also be in an active state or the test impulse is carried out in the active state of the reverse osmosis system. The value of the system-specific parameter is then set as part of the test impulse, whereby the test impulse is terminated when the system reaction has reached a stationary end value or has expired after a predefined time.

The test pulses are preferably carried out during the above-described pre-operation and/or intermediate operation.

The control and regulation unit of the reverse osmosis system advantageously has an interface that allows a user to set the scaling factors and the error measure in order to incorporate the local requirements for the control loop. For this purpose, the control and regulation unit can have a user interface in the form of a GUI and/or a data interface.

In a preferred embodiment, the method is carried out at regular intervals so that changes to the reverse osmosis system and/or the connected devices can be taken into account.

The advantages of the present disclosure lie in particular in the fact that the process enables an optimized operation of a reverse osmosis system that is adapted to the specific conditions and requirements.

### BRIEF DESCRIPTION OF THE DRAWINGS

An example is explained in more detail in the drawings that contain a highly schematized representation.

FIG. 1 shows a reverse osmosis system in a preferred embodiment;

FIG. 2 shows a flow diagram of a method for operating a reverse osmosis system in a first preferred embodiment;

FIG. 3 shows a flow diagram of a method for operating a reverse osmosis system in a second preferred embodiment; and

FIG. 4 shows an error measure of the method according to FIG. 3.

The same parts are provided with the same reference signs in all figures.

### DETAILED DESCRIPTION

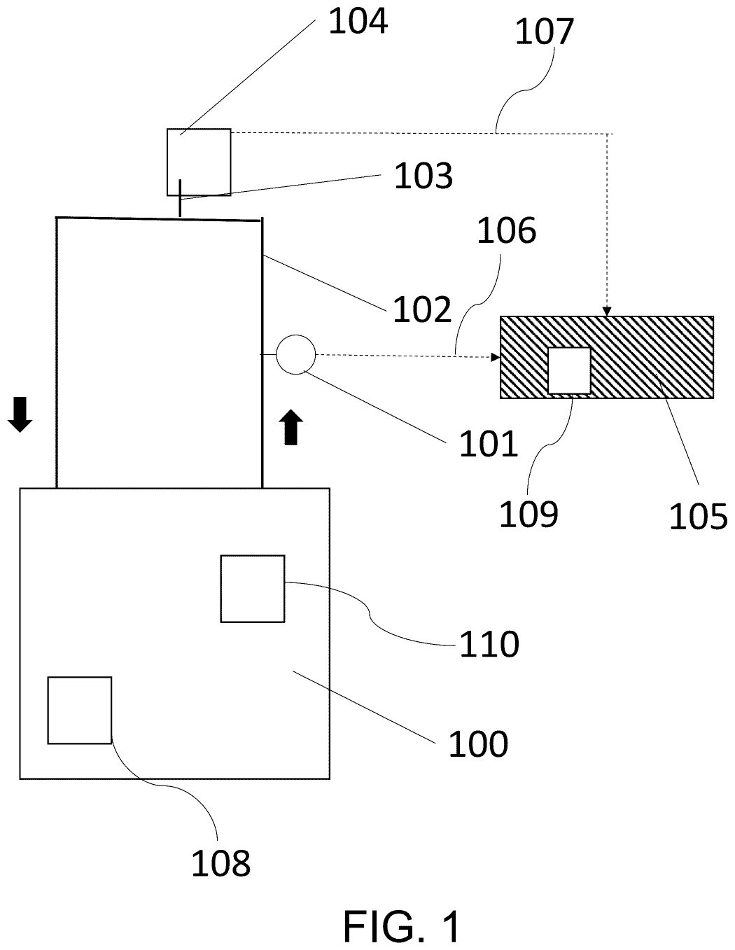

FIG. 1 shows a reverse osmosis system 100, to which a ring line 102 with a first tapping point 103 is hydraulically connected. The reverse osmosis system 100 has a control and regulating unit 105. The control and regulating unit 105 is preferably a control and regulating unit 105 that is installed in the reverse osmosis system 100.

A pressure sensor 101, which measures the pressure in the ring line 102, is hydraulically connected to the ring line 102. The pressure sensor 101 is connected on the signal input side to the control and regulating unit 105 by means of a data line 106. A test device 104 is connected to the first tapping point 103, which is located downstream of the reverse osmosis system 100, and this test device is also connected on the signal input side to the control and regulating unit 105 by means of a data line 107. The control and regulating unit 105 has a PI controller 109, which is designed to control the reverse osmosis system 100, in particular its pump(s), on the basis of recorded sensor data in a control loop.

The reverse osmosis system 100 has at least one membrane module 110 for filtering liquid. The filtered liquid, the permeate, is separated from concentrate in the membrane module 110. The reverse osmosis system 100 conveys the permeate into the ring line 102. For this purpose, it has a booster pump 108.

The pressure sensor 101 is either located in the reverse osmosis system 100 (not shown) or, as shown in FIG. 1, downstream of it at the beginning of the ring main 102. The test device 104 consists of at least one coupling for connecting to the first tapping point 103 and a pressure sensor.

The maximum pressure in the ring line flow is essentially influenced by two factors. The first factor is the ring line material. For example, the material PEX allows a maximum pressure of 10 bar, while stainless steel allows a maximum pressure of 25 bar, whereby in general such maximum pressure values are dependent on the material thickness and temperature.

A dialysis machine connected to the first terminal unit 103, for example, is the second factor and allows a maximum pressure of 6 bar, for example. Dialysis machines thus only allow a lower pressure than the corresponding ring line. However, it is not clearly defined where the first dialysis machine is positioned. This means that the pressure loss up to the first tapping point 103 can be applied to 6 bar.

The method according to the present disclosure is used to determine a maximum threshold value for the ring main pressure.

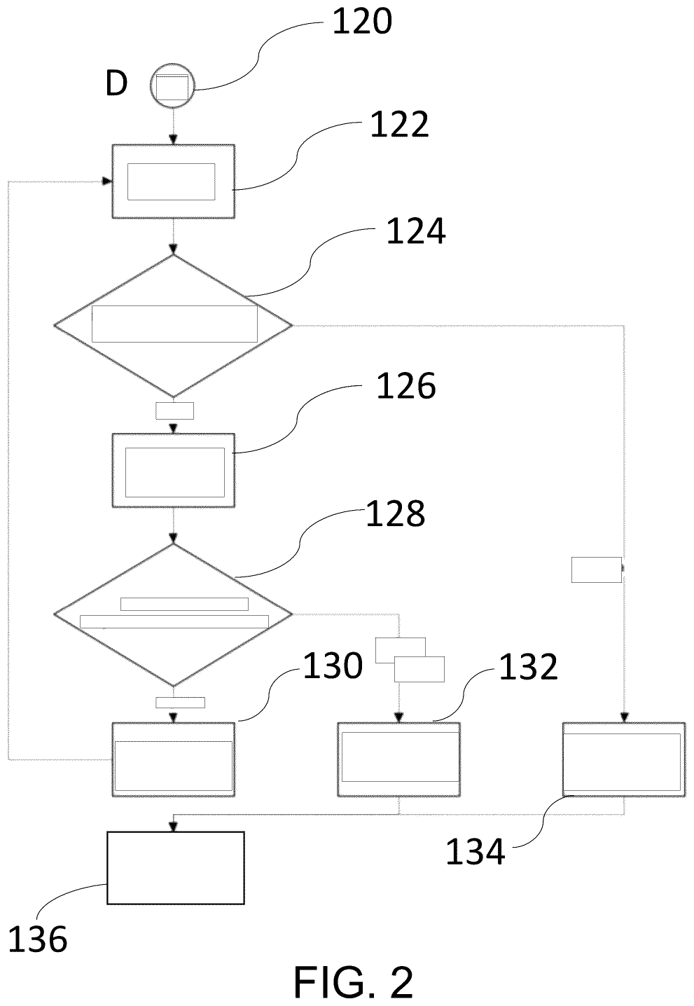

The method shown in FIG. 2 starts at a start 120. The speed D of the booster pump 108 is selected as the system-specific parameter. An optimized system reaction of the reverse osmosis system is defined as a function of this speed. In the present case, an optimized system reaction is present if the first pressure first reaches a first pressure threshold value or if the second pressure first reaches a second pressure threshold value.

In a block 122, the primary pressure in the ring line 102 is evaluated.

In a decision 124, it is checked whether the maximum primary pressure has been reached.

If this is not the case, the pressure measured by the test device 104 is evaluated in a block 126.

A decision 128 is used to check whether the pressure measured by the test device 104 has reached the maximum dialysis machine pressure (6 bar in the present example).

If the maximum dialysis machine pressure is not reached, the speed D of the pressure booster pump 108 is increased in a block 130 and the process continues in block 122. To increase the pump speed, the pressure booster pump 108 is controlled by the control and regulating unit 105.

If the maximum dialysis machine pressure is reached, the process continues in a block 132. The optimized system reaction is available. The pressure threshold value is set to the current pressure value of the pressure in the ring line 102, which is measured by the pressure sensor 101. The process is continued in a block 136 in which the reverse osmosis system 100 is operated with the determined pressure threshold value PS.

If the check in decision 124 shows that the maximum ring pressure has been reached, the process branches to a block 134 in which the pressure threshold value is set to the maximum ring pressure measured by the pressure sensor 101. The optimized system response has been obtained. The procedure is continued in a block 136 in which the reverse osmosis system 100 is operated with the determined pressure threshold PS.

In summary, the booster pump 108 of the reverse osmosis system 100 successively increases the speed D of the booster pump 108 until either the pressure sensor 101 reaches the pressure of 10 bar or the test device 104 reaches the pressure of 6 bar.

The stated pressure values 6 bar and 10 bar are based on the defined interfaces and/or permitted connectable devices. The pressures are therefore to be understood as an illustrative example and not as generally valid values. The method described in connection with FIG. 2 is carried out during the determination of the threshold value with a sufficient sampling rate, or the successive increase in pump speed is carried out slowly enough to determine the pressure threshold value as accurately as possible. In addition, safety limit values, e.g. 0.2 bar below the determined value, can be applied so that the system can compensate for measurement tolerances. In this way, overshooting of the controller or a pressure peak can also be taken into account in a safety-oriented manner.

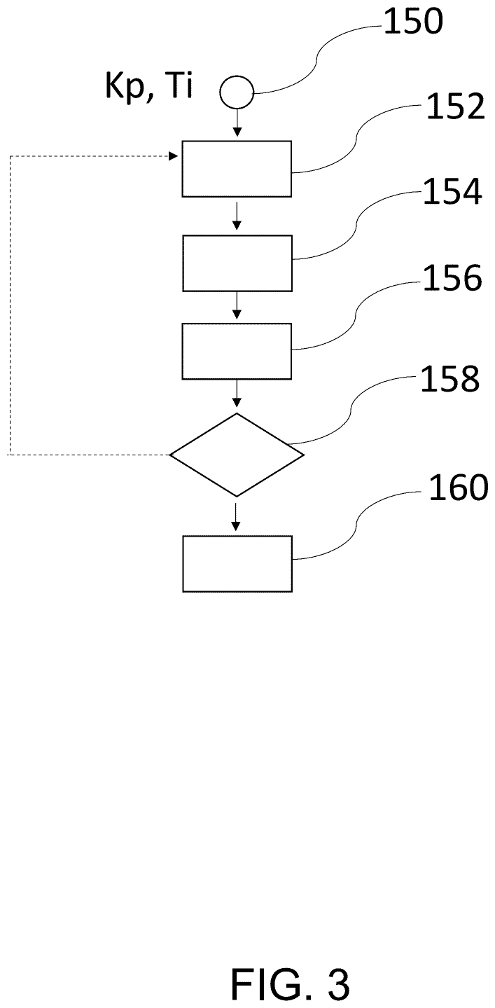

A method in a second preferred embodiment is described in connection with FIG. 3, in which optimized control parameters of the PI controller 109 are determined.

Control loops function optimally when they react quickly to changes and exhibit a low overshoot/undershoot behavior. Depending on which controller type (PID, Pl2D, PI, PD, State Space, etc.) or which system complexity is present, there are a variety of parameters that influence the behavior.

By gradually changing one or more parameters and measuring the optimized system reaction, an incremental improvement/optimization of the control of the reverse osmosis system 100 by the PI controller 109 can be achieved.

The PI controller 109 has two variable parameters, namely Kp and Ti. In this context, Kp is the controller amplification in the known sense, which specifies how strongly the PI controller reacts to the control difference, and Ti is the reset time, which denotes the time that must elapse for the I-component to be equal to the P-component. The procedure can also be used for systems of higher or lower complexity with regard to parameters/degrees of freedom. Changing the parameters Kp and Ti influences the characteristics of the PI controller 109.

In the present example, three properties or characteristic variables that influence the characteristics of the PI controller 109 are selected, namely the rise time [in seconds], the settling time [in seconds] and overshoot/undershoot [in percent].

From these properties, an error measure J is generated, which is used to evaluate the quality of the characteristic. The error measure J is calculated as follows

J=a*rise time+b*settling time+c*overshoot/undershoot (“*” denotes multiplication).

The factors a, b, and c are scaling factors that can be used to prioritize the respective property in the error measure J. The scaling factors or priority numbers a, b, c can thus be used to adjust the error according to importance (e.g. rise time is more important than settling time). Furthermore, properties with different dimensions (here percent and seconds) can be scaled in such a way that they reflect the importance for the overall characteristics.

At the start, the above-mentioned parameters Kp and Ti of the controller are selected as system-specific parameters. The optimized system reaction is defined as a minimum of the above-mentioned error measure J, which depends on these characteristic variables, which in turn depend on the parameters Kp and Ti.

In a block 152, at least one of the parameters Kp, Ti is varied and a test pulse is applied to the system of the reverse osmosis system 100. In the case of the test pulse, the system is started in the off state or a stationary state and adjusted to the setpoint. As soon as the process value has reached the stationary end value, the test pulse is terminated. In an alternative version of the test impulse, the system can already be active and the setpoint is changed.

In the example shown, a step in the setpoint is used to determine the error measure. The system initially “rests” (control output=0%; setpoint deviation=0). Subsequently, the setpoint is to be reached (e.g. 3 bar). The step change in the setpoint causes the setpoint deviation to increase and the control characteristic can be observed.

It is preferable not to increment/decrement both parameters according to a static principle, as this increases the duration of the parameter determination.

Varying the two parameters Kp, Ti is best done using a gradient step method. This automatically achieves convergence to a minimum. In addition to the gradient step method, an adaptive step size control is used in a preferred embodiment, which allows the minimum to be found more quickly. Instead of a fixed increment/decrement of the control values, the step size of the parameters is adapted to the rate of change of the error measure. Thus, only small changes are made to the parameters when the error measure changes only slightly, whereas large changes to the parameters are made when the quality measure changes significantly.

The rise time, the settling time and the overshoot/undershoot are measured in a block 154. The error measure J is calculated from these variables in a block 156 using the formula shown above.

In a decision 158, it is checked whether a predefined criterion for reaching the minimum has been achieved, which is the achievement of the optimized system reaction within the predefined threshold range, no further reduction of the error is possible or the rate of change of the error is smaller than a predefined threshold value.

If this is not the case, the process returns to block 152. If the criterion is fulfilled, the process continues to block 160, in which the current controller parameters are defined as optimized control parameters and the reverse osmosis system is operated with the optimized values of the controller parameters Kp, Ti.

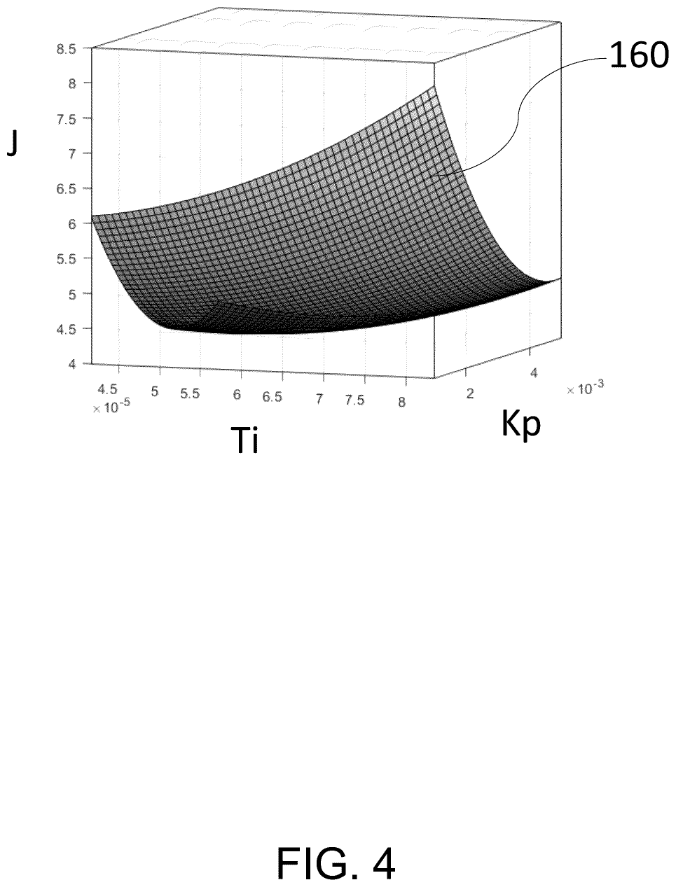

In FIG. 4, the error measure J is plotted against the control parameters Ti and Kp in an interpolated representation (the parameter space is scanned in discrete steps). This results in a paraboloid which has a minimum. To determine the optimal control parameters, the minimum of the paraboloid can be calculated. This indicates the optimal control parameters for the defined error measure J. In a preferred embodiment of the method, the error measure J is averaged. To do this, we prefer to increase the quantity of test cycles for the same parameter set (e.g. three test cycles for the same values) and calculate the mean value over these measurements.

Since the conditions for determining the visual acuity value are dynamically dependent on the connected devices and environmental conditions, the user should have the option of defining the conditions. This can be done either by entering values or selecting permitted devices via an enum (stainless steel ring line=25 bar; PEX=10 bar; dialysis machine=6 bar; etc.).

Instead of the pressure sensor 101 according to FIG. 1, a parameterizable pressure sensor with switch characteristics can also be used. In this case, the switch should be set to the determined value by the control and regulating unit 105 via an IO-Link, for example.

The test device 104 according to FIG. 1 can also be a dialysis machine or other regular consumer, which uses a network connection to transmit the pressure at the water inlet to the reverse osmosis system 100 or to the control and regulating unit 105, or also transmits the corresponding data to a cloud that can be accessed by the control and regulating unit 105.

The procedure described above involves a one-time execution to determine the optimized parameter values. The determination of threshold values can also be carried out iteratively every hour, day or month using network technology. This has the advantage that if the entire system is converted, i.e. the location of the first tapping point 103 is changed, the threshold value is also adjusted. This requires a network connection including pressure measurement of all active tapping points in the system.

The user should be able to influence the priority numbers or scale factors and the calculation of the error measure or quality measure in order to integrate the local requirements for the control loop.

### LIST OF REFERENCE SIGNS

100 reverse osmosis system
101 pressure sensor
102 ring line
103 first tapping point
104 test device
105 control and regulating unit
106 data line
107 data line
108 booster pump
109 PI controller
110 membrane module
120 start
122 block
124 decision
126 block
128 decision
130 block
132 block
134 block
136 block
150 start
152 block
154 block
156 block
158 decision
160 paraboloid
J error measurement
a scaling factor
b scaling factor
c scaling factor
D speed
PS pressure threshold value
Kp controller gain
Ti reset time

1. A method for operating a reverse osmosis system that comprises at least one booster pump, at least one membrane module and a ring line with at least one tapping point, the method comprising the steps of:
a) selecting at least one system-specific parameter of the reverse osmosis system and defining an optimized system reaction of the reverse osmosis system as a function of the at least one system-specific parameter of the reverse osmosis system;
b) iteratively modifying the at least one system-specific parameter of the reverse osmosis system;
c) measuring a system response of the reverse osmosis system to the at least one system-specific parameter of the reverse osmosis system after modification;
d) upon reaching the optimized system reaction within a predetermined threshold range, defining at least one optimized operating parameter and/or threshold value; and
e) operating the reverse osmosis system with this at least one optimized operating parameter and/or threshold value.

2. The method according to claim 1, further comprising the step of c1) adjusting a changing of the at least one system-specific parameter of the reverse osmosis system in order to achieve a system response which is closer to the optimized system reaction.

3. The method according to claim 2 wherein step c1) is carried out by carrying out a gradient step method for changing at least one characteristic variable in steps b) and c1).

4. The method according to claim 3, in which a variable step size, dependent on a rate of change of an error measure, is used for iteratively changed parameters in the gradient step method.

5. The method according to claim 2, wherein step c1) is carried out iteratively with steps b) and c).

6. The method according to claim 1, wherein the optimized system reaction corresponds to a threshold value of at least one measurable variable in the reverse osmosis system.

7. The method according to claim 6, wherein a first pressure in or downstream of the reverse osmosis system at a start of the ring line is measurable, and wherein a second pressure at at least one tapping point is measurable, and wherein
in step a) a speed of the at least one booster pump is selected as the at least one system-specific parameter, and the optimized system reaction is present when the first pressure first reaches a first pressure threshold value or when the second pressure first reaches a second pressure threshold value, and
in step b) a rotational speed of the at least one booster pump is increased iteratively, and
in step c) the first pressure and the second pressure are measured, and
in step d), when the optimized system reaction is reached within the predetermined threshold range, a current pressure is set as a maximum pressure threshold value of the reverse osmosis system at the start of the ring line.

8. The method according to claim 1, wherein the optimized system reaction corresponds to an extremum of an error measure dependent on the at least one system-specific parameter.

9. The method according to claim 8, wherein the optimized system reaction corresponds to a minimum of an error measure dependent on the at least one system-specific parameter.

10. The method according to claim 9, wherein the reverse osmosis system has at least one control loop with at least one controller with at least one controller parameter, and wherein
in step a) characteristic variables of the at least one controller are selected as system-specific parameters, and the optimized system reaction corresponds to a minimum of an error measure which depends on these characteristic variables, and
in step b) at least one of the characteristic variables is changed in an iteration, and
in step c) the error measure is determined based on the system response, and
in step d) when the optimized system reaction is reached within the predetermined threshold range, the at least one controller parameter is defined as an optimized controller parameter.

11. The method according to claim 10, in which the error measure is a weighted sum of the characteristic variables.

12. The method according to claim 10, wherein the characteristic variables are a rise time, a transient time and an overshoot/undershoot.

13. The method according to claim 10, wherein the at least one controller is a PI controller and wherein the at least one controller parameter comprises Kp and Ti.

14. The method according to claim 1, wherein steps a) to d) are carried out in a pre-operation and/or intermediate operation in which a consumer does not withdraw liquid at the at least one tapping point.

15. The method according to claim 1, wherein in step c) a test pulse is applied to the reverse osmosis system to measure the system response.

16. The method according to claim 15, wherein:
when the test pulse is applied, the reverse osmosis system is started from an off state or an idle state and is set to a value of the at least one system-specific parameter, and
the test pulse is ended when the system response has reached a stationary end value or has elapsed after a predefined time.

17. The method according to claim 15, wherein:
reverse osmosis is in an active state during the test pulse,
a value of the at least one system-specific parameter is set during the test pulse, and
the test pulse is ended when the system response has reached a stationary end value or has elapsed after a predefined time.
